(19) United States
(12) Patent Application Publication
(10) Pub. No.: US 2015/0051401 A1
(43) Pub. Date: Feb. 19, 2015

(54) PROCESS FOR PREPARING LACTAMS

(71) Applicant: RHODIA OPERATIONS, Aubevilliers (FR)

(72) Inventors: Roland Jacquot, Francheville (FR); Philippe Marion, Vernaison (FR)

(21) Appl. No.: 14/395,994

(22) PCT Filed: Apr. 25, 2013
(86) PCT No.: PCT/EP2013/058591

§ 371 Date: Oct. 21, 2014

(30) Foreign Application Priority Data
Apr. 27, 2012 (FR) 1253946

(51) Int. Cl. C07D 211/76; C07D 207/267

(52) U.S. Cl. CPC C07D 211/76; C07D 207/267
USPC 546/243; 548/543

(57) ABSTRACT

The present invention relates to a method for preparing lactams using heterogeneous catalysis by hydrogenating at least one compound of the following formula (I), where A is a radical of the following formula (I′) or (II′): —CH(R₁)—CH(R₂)— (I′); or —CH(R₁)—CH(R₂)—CH(R₃)— (II′); where R₁, R₂ and R₃ are, independently from each other, H, OH, an alkyl radical, or a cycloalkyl radical; and R is H or a straight or branched alkyl radical having 1 to 20, preferably 1 to 10, and more preferably 1 to 4 carbon atoms. Said method is carried out at a pressure of less than 60 bars, preferably 10 to 50 bars, in the presence of a solid hydrogenation catalyst including at least two metals selected from the group of noble metals and transition metals, and an inert substance used as a support, wherein said compound of formula (I) can be used alone or as part of a mixture.

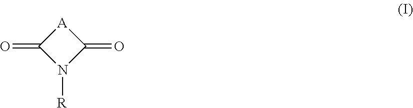

The present invention relates to a process for preparing lactams from cyclic imide compounds.

Lactams are cyclic amides well known to those skilled in the art. Lactams can, for example, be prepared by cyclization of an amino acid such as lysine. They can also be prepared by reacting an aminonitrile with water in the presence of a catalyst in order to carry out a cyclizing hydrolysis of the aminonitrile to give a lactam.

As a process for preparing lactams, mention may also be made of Beckmann rearrangement catalyzed by a strong acid which corresponds to the conversion of oxime into lactam, the oxime being obtained by condensation of cycloalkanone with NH₂OH, hydroxylamine.

Lactams are used in various fields, and in particular in the production of polyamides. Lactams can also be used as plasticizers or else as solvents, for example for N-alkyllactams such as NMP, or as intermediates for pharmaceutical and agrochemical product syntheses.

There is therefore at the current time a need to provide an efficient process for preparing lactams.

The aim of the present invention is to provide a novel process for preparing lactams from cyclic imides by heterogeneous catalysis. Heterogeneous catalysis reactions are the most common catalysis reactions used and consist in using a catalyst which is insoluble in the reaction medium; it can therefore easily be recovered. The catalyst is generally supported on an inert support.

Homogeneous catalysis reactions are described in documents WO2005/0501907 and Aoun et al., vol 44, no. 13, p. 2021-2023.

WO2005/0501907 describes the preparation of N-methylpyrrolidone by hydrogenation of N-methylsuccinimide in the presence of a catalyst which is soluble in the reaction medium. The catalyst used is ruthenium bonded to an organic ligand of phosphine type; in particular, Ru-acetylacetonate is used as a precursor. This type of ligand has drawbacks of health and environmental type.

The aim of the present invention is also to provide lactams with satisfactory yields, in particular greater than 50%, and preferably greater than 75%, or even 80% or 90%.

Processes for preparing a lactam by hydrogenation of an imide by heterogeneous catalysis in the presence of a catalyst comprising a single metal are known. The document Patton et al., J. of the Chem. Soc., Vol 1, p. 1611-1615 describes, for example, the use of ruthenium on carbon or palladium on carbon, without success in the latter case. Document WO2004/058708 describes the use of a monometallic catalyst at high pressure (of about 105 bar).

Thus, the present invention relates to a process for preparing a lactam, by hydrogenation of at least one compound of formula (I) below:

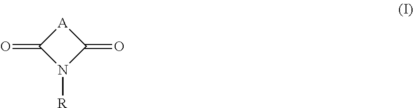

in which:
A represents a radical of formula (I′) or (II′) below:

—CH(R₁)—CH(R₂)— (I′)

or

—CH(R₁)—CH(R₂)—CH(R₃)— (II′)

in which:
R₁, R₂ and R₃ represent, independently of one another, H, OH, an alkyl radical or a cycloalkyl radical;
R₁ and R₂ can be linked together to form, with the carbon atoms which bear them, an aliphatic ring comprising from 4 to 6 carbon atoms;
R₂ and R₃ can be linked together to form, with the carbon atoms which bear them, an aliphatic ring comprising from 4 to 6 carbon atoms; and
R represents H or a linear or branched alkyl radical comprising from 1 to 20, preferably from 1 to 10 and preferentially from 1 to 4 carbon atoms;

said process being carried out at a pressure of less than 60 bar, preferably ranging from 10 bar to 50 bar, in the presence of a solid hydrogenation catalyst comprising at least two metals selected from the group of noble metals and transition metals, and an inert substance as support;

it being possible for said compound of formula (I) to be alone or in a mixture.

In the context of the invention, the term “lactam” denotes cyclic amides that can be represented by formula (II) below:

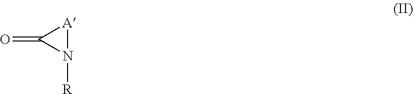

R being as defined above in formula (I) and A′ representing a radical of formula —CH(R₁)—CH(R₂)—CH₂— or —CH(R₁)—CH(R₂)—CH(R₃)—CH₂—, R₁, R₂ and R₃ being as defined above in formulae (I′) and (II′).

According to the present invention, the “alkyl” radicals represent straight-chain or branched-chain saturated hydrocarbon-based radicals comprising from 1 to 10 carbon atoms, preferably from 1 to 5 carbon atoms (they can typically be represented by the formula CₙH₂ₙ₊₁, n representing the number of carbon atoms). When they are linear, mention may in particular be made of methyl, ethyl, propyl, butyl, pentyl, hexyl, octyl, nonyl and decyl radicals. When they are branched or substituted with one or more alkyl radicals, mention may in particular be made of isopropyl, tert-butyl, 2-ethylhexyl, 2-methylbutyl, 2-methylpentyl, 1-methylpentyl and 3-methylheptyl radicals.

The “cycloalkyl” radical is a nonaromatic, saturated monocyclic, bicyclic or tricyclic hydrocarbon-based radical preferably comprising 5 or 6 carbon atoms, such as, in particular, cyclopentyl or cyclohexyl.

The process according to the invention can be carried out for a compound of formula (I) alone or for a mixture of various compounds of formula (I).

Thus, the compound subjected to the hydrogenation step can be a mixture of compounds of formula (I), with it being possible for A and/or R to be different.

The process of the invention consists in hydrogenating one of the carbonyl functions of the imide (I).

Thus, the resulting lactam corresponds to the formula below:

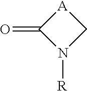

Since this carbonyl function hydrogenation step is not selective, either of the carbonyl functions is hydrogenated.

The starting compound can be represented by the formula below:

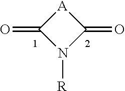

In the context of the invention, it is the carbonyl function 1 or the carbonyl function 2 which is hydrogenated, thereby making it possible to obtain one of the compounds below:

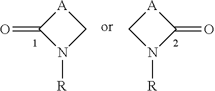

Depending on the nature of A, as explained hereinafter, these two compounds can be identical or different.

Thus, depending on the nature of A, the lactam obtained can be a mixture of several compounds, namely positional isomers.

More particularly, when A is not a symmetrical group, the product obtained is a mixture of the positional isomers.

As indicated above, A corresponds to formula (I′) or (II′) as defined above.

When R₁ and R₂ are different in formula (I′) or R₁ and R₃ are different in formula (II′), then the lactam obtained will be in the form of a mixture comprising the positional isomers, i.e. a mixture of the lactams obtained by hydrogenation of each of the carbonyl functions.

More particularly, when A corresponds to formula (I′), in which either one of R₁ and R₂ is an alkyl radical (the other being H) or R₁ and R₂ are different alkyl radicals, then the lactam obtained will be in the form of a mixture comprising the positional isomers, i.e. a mixture of the lactams obtained by hydrogenation of each of the carbonyl functions.

More particularly, when A corresponds to formula (II′), in which either R₁ or R₃ is an alkyl radical (the other two being H), or R₁, R₂ and R₃ are alkyl radicals, R₁ and R₃ being different, or R₁ and R₂ are alkyl radicals (R₃ being H), which may be identical or different, or R₂ and R₃ are alkyl radicals (R₁ being H), which may be identical or different, or R₁ and R₃ are different alkyl radicals (R₂ being H), then the lactam obtained will be in the form of a mixture comprising the positional isomers, i.e. a mixture of the lactams obtained by hydrogenation of each of the carbonyl functions.

According to one preferred embodiment, the hydrogenation process according to the invention is carried out in the absence of solvent.

This embodiment makes it possible to work in a more concentrated medium. Such a process makes it possible to be more competitive from an industrial point of view.

Thus, according to this embodiment, the process consumes less energy and generates fewer effluents linked to the presence of solvent, compared with the processes with solvent.

According to one preferred embodiment, the hydrogenation process according to the invention is carried out in the liquid phase. The process of the invention can therefore be carried out in conventional hydrogenation reactors.

As indicated above, the starting imide compound, subjected to the hydrogenation process, can be a single compound or a mixture of several compounds of formula (I) as defined above.

According to one embodiment, the imide compounds of the process of the invention are compounds of formula (I), in which A corresponds to formula (I′) or (II′) as defined above, each of R₁, R₂ and R₃ representing H or an alkyl radical, in particular a (C₁-C₄)alkyl radical.

According to one embodiment, in formula (I) as defined above, A is a radical of formula —CH₂—CH₂—CH(R′)—, R′ representing a (C₁-C₄)alkyl radical, and preferably methyl or ethyl.

As specific examples of group A according to the invention, mention may be made of ethylene (—CH₂—CH₂—) or propylene (—CH₂—CH₂—CH₂—), or else 1-methylpropylene (—CH₂—CH₂—CH(CH₃)—).

Particularly preferably, the selection will be made from the following radicals: ethylene (—CH₂—CH₂—), propylene (—CH₂—CH₂—CH₂—), ethylethylene (—CH(Et)—CH₂—) and 1-methylpropylene (—CH₂—CH₂—CH(CH₃)—), and mixtures thereof.

According to one preferred embodiment, A represents a —CH₂—CH₂—CH(CH₃)— radical.

According to one embodiment, in formula (I), R is H or Me.

Preferably, in formula (I), R is H.

The present invention therefore also relates to the preparation of lactams by hydrogenation of a mixture of compounds of formula (I).

According to one embodiment, the invention relates to the preparation of lactams by hydrogenation of a mixture comprising the following compounds:

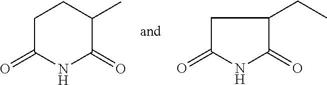

namely of a mixture of MGI and of ESI, respectively.

The present invention therefore also relates to a process as defined above, for preparing a mixture of lactams of formulae (II-5) and (II-6) below:

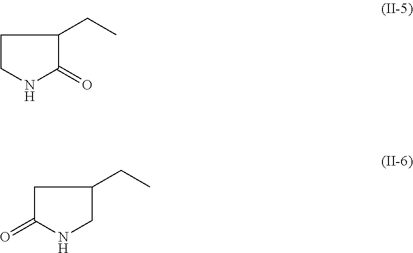

by hydrogenation of the imide of formula (I-3) below:

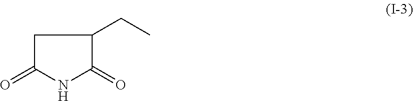

The present invention also relates to a process as defined above, for preparing a mixture of lactams of formulae (II-1) and (II-2) below:

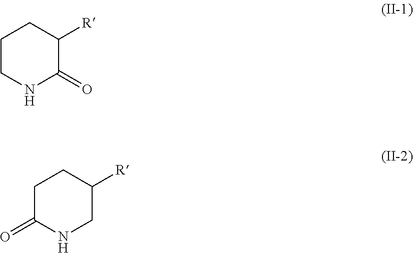

by hydrogenation of the imide of formula (I-1) below:

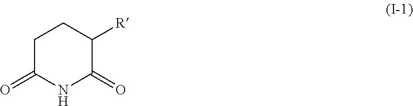

R′ representing a (C₁-C₄)alkyl radical, and preferably methyl or ethyl.

The present invention also relates to a process as defined above, for preparing a mixture of lactams of formulae (II-3) and (II-4) below:

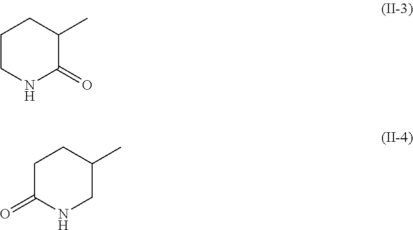

by hydrogenation of the imide of formula (I-2) below:

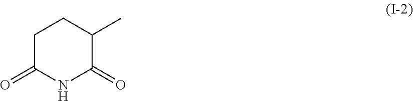

According to one embodiment, the starting imide of formula (I) can be methylglutarimide (MGI) obtained from methylglutaronitrile (MGN), or from a mixture of dinitriles resulting from the process for producing adiponitrile by double hydrocyanation of butadiene. This mixture preferably corresponds to the distillation fraction making it possible to separate the branched dinitriles (methyl-2-glutaronitrile, ethyl-2-succinonitrile) from the adiponitrile.

This dinitrile mixture generally has the following weight composition:
methyl-2-glutaronitrile: between 70% and 95%, preferably between 80% and 85%;
ethyl-2-succinonitrile: between 5% and 30%, preferably between 8% and 12%; and
adiponitrile: between 0% and 10%, preferably between 1% and 5%, the rest to 100% corresponding to various impurities.

The starting imide of formula (I), when it is in particular methylglutarimide (MGI), can be obtained from methylglutaronitrile (MGN), or from a mixture of dinitriles as described above, for example according to a process of reacting the MGN or the dinitrile mixture with an acid, as described in international application WO2011/144619. It can also be obtained according to a process of hydrolysis of the MGN or of the dinitrile mixture, in the presence of water, which corresponds to the first step of the process described, for example, in international application WO2009/056477.

The process of the invention is carried out at a pressure of less than 60 bar, preferably less than 50 bar, in order to avoid hydrogenation of the two carbonyl functions, which would prevent lactams from being obtained.

According to one embodiment, the process of the invention is carried out at a pressure ranging from 10 bar to 50 bar and preferably at a pressure ranging from 10 to 40 bar, in particular from 20 to 40 bar and preferentially equal to 20 bar.

According to one preferred embodiment, the pressure is from 20 to 25 bar. The process of the invention therefore makes it possible to work at low pressures, which is particularly advantageous from an industrial point of view.

Preferably, the process of the invention is carried out at a temperature above the melting point of the imides.

According to one embodiment of the process of the invention, the hydrogenation is carried out at a temperature greater than or equal to 105° C. and preferably less than 230° C.

It is preferable to work at temperatures of less than 230° C. in order to avoid polymerization reactions.

According to one advantageous embodiment, the hydrogenation is carried out at a temperature ranging from 150° C. to 220° C. and preferably equal to 200° C.

The process of the invention is carried out in the presence of a solid hydrogenation catalyst.

The term “solid hydrogenation catalyst” denotes any solid catalyst well known to those skilled in the art for catalyzing hydrogenation reactions.

This catalyst can be free or attached to an inert support, in particular to carbon, silica or alumina.

According to one embodiment, the hydrogenation catalyst used in the context of the invention comprises a mixture of metals selected from the group of noble metals and transition metals, and optionally an inert substance as support.

According to one embodiment, the hydrogenation catalyst used in the context of the invention comprises a mixture of two or three metals selected from the group of noble metals and transition metals, and optionally an inert substance as support.

According to one embodiment, the hydrogenation catalyst comprises an inert substance supporting the metals as defined above.

As indicated previously, the catalyst according to the invention can comprise a support on which a mixture of metal is supported or can be a mixture of several metals, it being possible for each of the metals to be supported independently of one another.

The term “noble metals” denotes a metal which withstands corrosion and oxidation. Among these metals, mention may be made of gold, silver and platinum.

The term “transition metals” denotes the elements which have an incomplete d sub-level or which can give a cation that has an incomplete d sub-level. In the context of the present invention, this term denotes the d elements which are not noble metals. The transition metals are selected from the elements of columns 3 to 12, with the exception of lutetium and lawrencium.

According to one embodiment, the hydrogenation catalyst as defined above comprises two metals M1 and M2, it being possible for each of the metals to be supported independently of one another or it being possible for the mixture M1+M2 to be supported.

Thus, according to one embodiment, M1 is supported by an inert substance S1 and M2 is supported by an inert substance S2, S1 and S2 being two distinct supports, of identical or different nature. In this case, the hydrogenation catalyst can also be denoted as a mixture of catalysts.

Thus, according to another embodiment, the mixture formed by the metals M1 and M2 is supported by a single inert substance S1.

In the context of the present invention, the hydrogenation catalyst can be a mixture of two metals selected from the group consisting of ruthenium, platinum, palladium, iridium and rhodium, said mixture being supported by an inert substance, in particular carbon.

According to one embodiment, the hydrogenation catalyst comprises, supported by carbon, ruthenium in a mixture with a metal selected from the group consisting of platinum, palladium, iridium and rhodium.

According to the invention, the hydrogenation catalyst can be in the form of a mixture comprising ruthenium supported by carbon and another metal as defined above, supported by carbon.

According to one preferred embodiment of the invention, the hydrogenation catalyst comprises a mixture of ruthenium and palladium, said mixture being supported by carbon.

In the context of the present invention, the weight content of catalyst is preferably from 1% to 10% relative to the total weight of compound(s) of formula (I). The weight content of catalyst corresponds to the weight content of the assembly formed by the metal and the support if it is present.

Preferably, the weight content of catalyst is 5% relative to the total weight of compound(s) of formula (I).

According to one preferred embodiment, the catalyst is a mixture of ruthenium and palladium supported on carbon, comprising from 2% to 7% of ruthenium and from 0.5% to 1.5% of palladium relative to the total weight of the catalyst, the rest by weight corresponding to the carbon support.

Preferably, the catalyst is a mixture of ruthenium and palladium supported on carbon, comprising 5% of ruthenium, 1% of palladium and 94% of carbon relative to the total weight of the catalyst.

The examples which follow make it possible to further illustrate the invention without limiting it.

### EXAMPLES

### Example 1

Hydrogenation of 3-methylglutarimide (MGI) with a Catalyst Mixture 20 g of MGI are placed in a stainless steel stirred autoclave, and 0.2 g of catalyst at 1% by weight Pd/carbon (Pd/C) and 0.8 g of catalyst at 5% by weight Ru/C are added. The autoclave is flushed twice with 20 bar of nitrogen and then with 3 times 20 bar of hydrogen. The autoclave is then placed at 15 bar and heating is carried out at 200° C. while stirring. A constant pressure of 20 bar is maintained in the autoclave throughout the duration of the reduction. After 12 hours of reaction the autoclave is brought back to ambient temperature and flushed with twice 20 bar of nitrogen. The reaction medium is then analyzed by gas chromatography.

The MGI conversion is 58% and the yield of mixture of the two lactams is 50%.

### Example 2

Hydrogenation of 3-methylglutarimide (MGI) with a Mixed Catalyst

20 g of MGI are placed in a stainless steel stirred autoclave, and 1.0 g of (5% by weight Ru +1% by weight Pd)/C catalyst is added. The autoclave is flushed twice with 20 bar of nitrogen and then with 3 times 20 bar of hydrogen. The autoclave is then placed at 15 bar and heating is carried out at 200° C. while stirring. A constant pressure of 20 bar is maintained in the autoclave throughout the duration of the reduction. After 12 hours of reaction the autoclave is brought back to ambient temperature and flushed with twice 20 bar of nitrogen.

The reaction medium is then analyzed by gas chromatography.

The MGI conversion is 100% and the yield of mixture of the two lactams is 92%; the presence of 3-methylpiperidine is not detected.

### Example 3

Hydrogenation of 3-methylglutarimide (MGI) at 40 Bar

20 g of MGI are placed in a stainless steel stirred autoclave, and 1.0 g of (5% by weight Ru +1% by weight Pd)/C catalyst is added. The autoclave is flushed twice with 20 bar of nitrogen and then with 3 times 20 bar of hydrogen. The autoclave is then placed at 15 bar and heating is carried out at 200° C. while stirring. A constant pressure of 40 bar is maintained in the autoclave throughout the duration of the reduction. After 4 hours of reaction the autoclave is brought back to ambient temperature and flushed with twice 20 bar of nitrogen. The reaction medium is then analyzed by gas chromatography.

The MGI conversion is 98% and the lactam mixture yield is 78%.

### Example 4

Hydrogenation of the Mixture of 3-ethylsuccinimide (ESI) and 3-methylglutarimide (MGI)

20 g of a mixture of imides which is composed of 87% of MGI and 11% of ESI are placed in a stainless steel stirred autoclave, and 1.0 g of (5% by weight Ru+1% by weight Pd)/C catalyst is added. The autoclave is flushed twice with 20 bar of nitrogen and then with 3 times 20 bar of hydrogen. The autoclave is then placed at 15 bar and heating is carried out at 200° C. while stirring. A constant pressure of 20 bar is maintained in the autoclave throughout the duration of the reduction. After 4 hours of reaction the autoclave is brought back to ambient temperature and flushed with twice 20 bar of nitrogen. The reaction medium is then analyzed by gas chromatography.

The imide conversion is 90% and the lactam mixture yield is 82%.

### Example 5

Hydrogenation of N-methyl-3-methylglutarimide (N—Me-MGI) with a Mixed Catalyst

20 g of N-methyl-MGI are placed in a stainless steel stirred autoclave, and 1.0 g of (5% by weight Ru+1% by weight Pd)/C catalyst is added. The autoclave is flushed twice with 20 bar of nitrogen and then with 3 times 20 bar of hydrogen. The autoclave is then placed at 15 bar and heating is carried out at 200° C. while stirring.

A constant pressure of 20 bar is maintained in the autoclave throughout the duration of the reduction. After 4 hours of reaction the autoclave is brought back to ambient temperature and flushed with twice 20 bar of nitrogen.

The reaction medium is then analyzed by gas chromatography. The N-methyl-MGI conversion is 40% and the yield of mixture of the two lactams is 29%.

### Example 6

Hydrogenation of N-octyl-3-methylglutarimide (N-Oc-MGI) with a Mixed Catalyst

20 g of N-octyl-MGI are placed in a stainless steel stirred autoclave, and 1.0 g of (5% by weight Ru+1% by weight Pd)/C catalyst is added. The autoclave is flushed twice with 20 bar of nitrogen and then with 3 times 20 bar of hydrogen. The autoclave is then placed at 15 bar and heating is carried out at 200° C. while stirring.

A constant pressure of 20 bar is maintained in the autoclave throughout the duration of the reduction. After 4 hours of reaction the autoclave is brought back to ambient temperature and flushed with twice 20 bar of nitrogen.

The reaction medium is then analyzed by gas chromatography. The MGI conversion is 32% and the yield of mixture of the two lactams is 25%.

1. A process for preparing a lactam, comprising: by of hydrogenating at least one compound of formula (I) below: 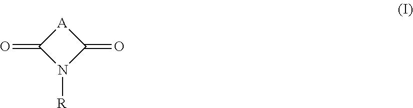
wherein:
A represents a radical of formula (I′) or (II′) below:
—CH(R₁)—CH(R₂)— (I′)
or
—CH(R₁)—CH(R₂)—CH(R₃)— (II′)
wherein:
R₁, R₂ and R₃ represent, independently of one another, H, OH, an alkyl radical or a cycloalkyl radical;
R₁ and R₂ can be linked together to form, with the carbon atoms which bear them, an aliphatic ring comprising from 4 to 6 carbon atoms;
R₂ and R₃ can be linked together to form, with the carbon atoms which bear them, an aliphatic ring comprising from 4 to 6 carbon atoms; and
R represents H or a linear or branched alkyl radical comprising from 1 to 20 carbon atoms;
said process being carried out at a pressure of less than 60 bar,
in the presence of a solid hydrogenation catalyst comprising at least two metals selected from the group of noble metals and transition metals, and an inert substance as support,
and wherein said compound of formula (I) may be alone or in a mixture.

2. The process of claim 1, wherein the hydrogenation is carried out in the absence of solvent.

3. The process of claim 1, wherein the hydrogenation is carried out in the liquid phase.

4. The process of claim 1, wherein R₁, R₂ and R₃ represent H or a (C₁-C₄)alkyl radical.

5. The process of claim 1, wherein A is a radical of formula —CH₂—CH₂—CH(R′)—, and R′ represents a (C₁-C₄)alkyl radical, and preferably methyl or ethyl.

6. The process of claim 1, wherein R is H.

7. The process of claim 1, wherein the hydrogenation is carried out at a temperature greater than or equal to 105° C.

8. The process of claims 1, wherein the lactam comprises a mixture of lactams of formulae (II-1) and (II-2) below: 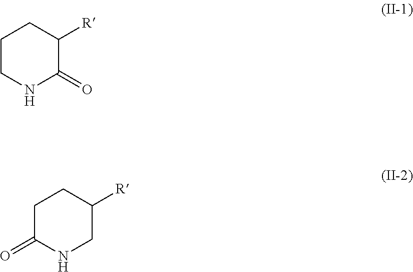
and wherein the at least one compound of formula (I) comprises an imide of formula (I-1) below: 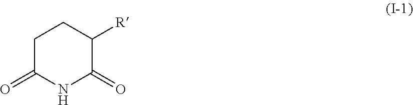
wherein R′ representing a (C₁-C₄)alkyl radical.

9. The process of claim 1, wherein the pressure is between 10 bar and 50 bar.

10. The process of claim 1, wherein R represents a linear or branched alkyl radical comprising from 1 to 10 carbon atoms.

11. The process of claim 1, wherein the hydrogenation catalyst is a mixture of at least two metals selected from the group consisting of ruthenium, platinum, palladium, iridium and rhodium, said mixture being supported by an inert substance.

12. The process of claim 1, wherein the hydrogenation catalyst comprises, supported by carbon, ruthenium in a mixture with at least one metal selected from the group consisting of platinum, palladium, iridium and rhodium.

13. The process of claim 1, wherein the hydrogenation catalyst comprises a mixture of ruthenium and palladium, and said mixture is supported by carbon.

14. The process of claim 1, wherein the weight content of catalyst is from 1% to 10%, relative to the total weight of compound(s) of formula (I).

15. The process of claim 5, wherein R′ is methyl or ethyl.

16. The process of claim 7, wherein the hydrogenation is carried out at a temperature of less than 230° C.

17. The process of claim 8, wherein R′ is methyl or ethyl.

18. The process of claim 10, wherein R represents a linear or branched alkyl radical comprising from 1 to 4 carbon atoms.

19. The process of claim 11, wherein the mixture is supported by carbon.
